(19) United States
(12) Patent Application Publication
(10) Pub. No.: US 2020/0108100 A1
(43) Pub. Date: Apr. 9, 2020

(54) NEURAL STEM CELL THERAPY FOR STROKE

(71) Applicant: Reneuron Limited, Bridgend (GB)

(72) Inventor: Keith Muir, Glasgow (GB)

(21) Appl. No.: 16/154,059

(22) Filed: Oct. 8, 2018

(51) Int. Cl. A61K 35/30; C12N 5/0797; A61P 9/00

(52) U.S. Cl. CPC A61K 35/30; A61P 9/00; C12N 5/0623

(57) ABSTRACT

The invention relates to the treatment of stroke using neural stem cells. In particular, the invention relates to functional improvements in stroke patients following treatment with CTX0E03 neural stem cells. In one aspect, the invention provides neural stem cells for use in a method of treating ischemic stroke, wherein a single dose of the cells is administered into the brain of a stroke patient having a modified NIHSS Motor Arm Score of 2 or 3, wherein the treatment improves motor function and alleviates disability within six months as determined by an increase of total ARAT score and/or a reduction in mRS of at least one category.

### FIELD

This invention relates to the treatment of stroke using neural stem cells. In particular, the invention relates to functional improvements in stroke patients following treatment with neural stem cells from the CTX0E03 cell line.

### BACKGROUND

One in six people worldwide will have a stroke in their lifetime. 15 million people worldwide suffer a stroke each year and 5.8 million people die from a stroke each year. In the UK alone, around 150,000 people suffer a stroke every year and a quarter of a million adults live with long-term disability as a result of stroke. About 40% of stroke survivors are left with a moderate to severe impairment according to the National Stroke Association and up to 75% of patients require help with daily activities.

The majority of strokes (80%) are termed “ischemic”. An ischemic stroke is caused by an interruption of blood flow to the brain resulting in cell death and damage. Following a stroke, most people experience at least some degree of recovery in the first 6 months, most rapidly and largely defined in the first few weeks. However, up to 30% of patients are left with persistent disability of some degree. Recovery after a stroke involves rehabilitation strategies such as physiotherapy. At present, there are no medical treatments that can repair the brain damage caused by a stroke.

Stem cells have the ability to self-renew and to differentiate into functionally different cell types. They have the potential to be a powerful therapeutic tool, for example in the growing field of Regenerative Medicine, in particular regenerative therapy requiring tissue replacement, regeneration or repair (Banerjee et al., 2011).

Neural stem cells (NSCs) are self-renewing, multipotent stem cells that generate neurons, astrocytes, and oligodendrocytes (Kornblum, 2007). The medical potential of neural stem cells is well-documented. Damaged central nervous system (CNS) tissue has very limited regenerative capacity so that loss of neurological function is often chronic and progressive. Neural stem cells (NSCs) have shown promising results in stem cell-based therapy of neurological injury or disease (Einstein et al., 2008). Implanting neural stem cells (NSCs) into the brains of post-stroke animals has been shown to be followed by significant recovery in motor and cognitive tests (Stroemer et al., 2009). It is not completely understood how NSCs are able to restore function in damaged tissues but it is now becoming increasingly recognised that NSCs have multimodal repairing properties, including site-appropriate cell differentiation, pro-angiogenic and neurotrophic activity, and immunomodulation promoting tissue repair by the native immune system and other host cells (Miljan & Sinden, 2009, Horie et al., 2011). It is likely that many of these effects are dependent on transient signalling from implanted neural stem cells to the host milieu, for example NSCs transiently express pro-inflammatory markers when implanted in ischemic muscle tissue damage which directs and amplifies the natural pro-angiogenic and regulatory immune response to promote healing and repair (Katare et al., Clinical-grade human neural stem cells promote reparative neovascularization in mouse models of hindlimb ischemia. Arteriosclerosis, Thrombosis and Vascular Biology, vol 34, no. 2, pp. 408-418). In chronic brain stroke, NSCs also have a substantial neurotrophic effect. For example, they promote the repopulation of the stroke-damaged striatal brain tissue with host brain derived doublecortin positive neuroblasts (Hassani, O'Reilly, Pearse, Stroemer et al., PLoS One. 2012; 7(11)).

Furthermore, on the basis of a large body of NSC restorative effects in animal models with chronic stroke, a Phase 1 clinical trial using neural stem cells was carried out by ReNeuron Limited (Surrey, UK), to test the safety of treatment of stably disabled stroke patients using its CTX0E03 conditionally-immortalised cortex-derived neural stem cells (CTX0E03 DP, Clinicaltrials.gov Identifier: NCT01151124; “PISCES”). This study is discussed by Kalladka et al., Lancet. 2016 Aug. 20; 388(10046):787-96, and concluded that single intracerebrally injected doses of CTX Drug Product up to 20 million cells induced no adverse events and were associated with improved neurological function in the majority of patients treated.

A Phase 2 trial of the CTX0E03 neural stem cells has recently completed. The study aimed to test the efficacy of these cells in patients with stable paresis of the arm following an ischemic stroke (NCT02117635; “PISCES II”). This study involved an injection into the brain of human neural stem cells (CTX0E03 DP). The study follows on from the previous study which was designed to assess the safety of different doses of CTX0E03 DP in ischemic stroke patients (2, 5, 10 and 20 million cells). The PISCES II study continued to assess safety; however it also assessed the efficacy of CTX0E03 DP, i.e., to see if the cells have an effect on disability. All participants in the study received the 20 million cell dose of CTX0E03 DP, to determine if this dose should be developed further as a potential treatment for stroke disability. The study enrolled 23 patients who had suffered a stroke between 2 and 10 months prior to CTX0E03 DP treatment.

Participants were assessed over a 1-year period using standard stroke assessment questionnaires and scales, such as the modified Rankin Scale of disability and the Barthel Index of activities of daily living, as well as tests of sensorimotor function, namely the Action Research Arm Test and the Fugl-Meyer assessment of motor performance, after which they will be monitored for life using a NHS central register.

A review of nearly 15 years of research behind the CTX0E03 cell line and its mode of action, together with implications for therapeutic potential in stroke disability, is provided by Sinden et al. Stem Cells Dev. 2017 Jul. 1; 26(13): 933-947.

There remains a need for effective therapies of stroke.

### SUMMARY

The present disclosure is based in part on surprising results observed in the “PISCES II” clinical trial. In particular, the inventors have surprisingly discovered that following administration of neural stem cells to stroke patients with some residual movement in a paretic arm, improvements are apparent in both the paretic arm and in the patient's general disability. That the improvements are not limited solely to arm function, goes significantly beyond the Primary Endpoints of the trial. This unexpected improvement in general disability is particularly surprising in the further subset of patients that began the trial with limited effort against gravity in the paretic arm.

A first aspect of the invention provides neural stem cells for use in a method of treating ischemic stroke, wherein a single dose of the cells is administered into the brain of a stroke patient having a NIHSS Motor Arm Score of 2 or 3, wherein the treatment improves motor function and alleviates disability within six months as determined by an increase of total ARAT score, and/or a reduction in the modified Rankin Scale of at least one category. In some embodiments, the functional improvements are observed within three months, or less.

In some embodiments, the neural stem cells are CTX0E03 cells, from the cell line deposited by ReNeuron Limited at the European Collection of Authenticated Cell Cultures (ECACC), Porton Down, UK and having ECACC Accession No. 04091601.

In other embodiments, the neural stem cell line is the “STR0C05” cell line, the “HPC0A07” cell line, or the neural stem cell line disclosed in Miljan et al., Stem Cells Dev. 2009.

In some embodiments, the neural stem cells are isolated or purified.

In some embodiments, the cells are administered intracerebrally at a single dose of between 16×10⁶ and 28×10⁶-cells, e.g., 20 million cells. In certain aspects, the improvement to motor function and alleviation of disability remains after 12 months. In some embodiments, the treatment improves function within one month as determined by a reduction of mRS of at least one category.

In some embodiments, the cells are administered within twelve months of the stroke occurring. In some embodiments, the cells are administered between 3 and 6 months after the stroke, or between 6 and twelve months after the stroke. Other periods between stroke and administration can be within four weeks from the stroke, within two months from the stroke, or between two and twelve months after the stroke.

In a further aspect, the invention provides a composition comprising a neural stem cell and a pharmaceutically acceptable excipient, carrier, or diluent, for use according to the first aspect. The composition can, in one embodiment, be formulated with excipients suitable for intracerebral administration.

### BRIEF DESCRIPTION OF THE DRAWINGS

FIG. 1 depicts the production of a stem cell drug product from a master cell bank.

FIG. 2 shows the PISCES II study schedule (by visit), with treatment shown at Visit 4 (Day 0=day of injection).

FIG. 3 shows the Recruitment and Patient flow for the PISCES II Study.

FIG. 4 depicts the demographics of the PISCES II study.

FIG. 5 shows the PISCES II Stroke characteristics at baseline.

FIG. 6 shows the PISCES II Responder Analysis: Primary and Secondary Measures.

FIG. 7 shows the ARAT efficacy results. In the ARAT (Grasp) Test #2, ≥2 point improvement is seen in 3 responders, responding at 3, 6, and 12 months and with no relapse back to a lower score. For Total ARAT, ≥6 point improvement is seen in 5 responders, responding at 1, 1, 3, 6, and 12 months.

FIG. 8 depicts Median Total ARAT Response by Baseline NIHSS. These Total ARAT Affected Limb results show improvements on ARAT observed in patients with residual movement of the affected arm (NIHSS 2 or 3 at baseline).

FIG. 9 shows the modified Rankin Scale (mRS) Distribution: mRS improvement (12 m or final measured) compared to baseline in 7 (6 by 1 category, 1 by 2 categories); mRS unchanged in 14, worse (1 category) in 2. NB: 12 month mRS outcome determined 1 week prior to suicide in one subject therefore only one death at month 12 shown.

FIG. 10 shows the mRS by Baseline NIHSS: Improvement in mRS is greatest in patients with residual movement of the affected arm (NIHSS 2 or 3 at baseline).

FIG. 11 shows Serious Adverse Events (SAEs) during follow-up.

### DETAILED DESCRIPTION

The present inventors have surprisingly identified that neural stem cell therapy of stroke patients with paresis of an arm and having minimal movement but no useful function of the paretic arm, results in improvements in the overall motor function of the patient. These improvements were not expected, and surpass the Primary Endpoint set for the trial, for improvements in the paretic arm.

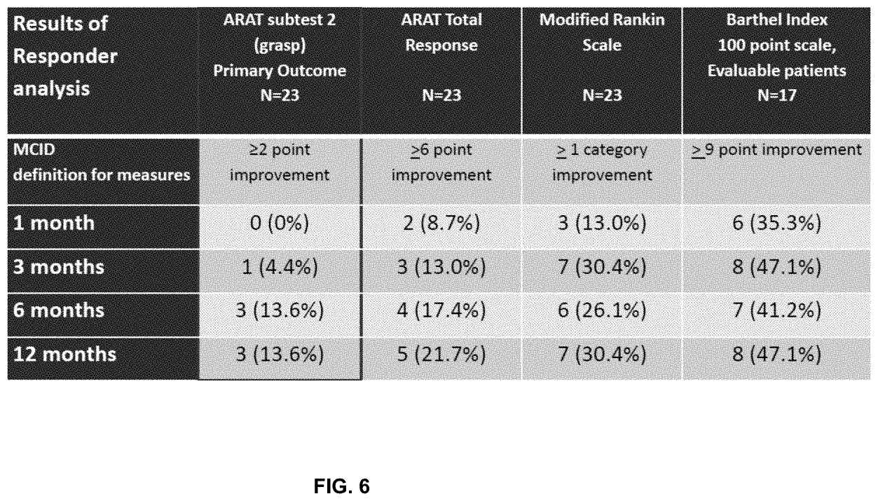

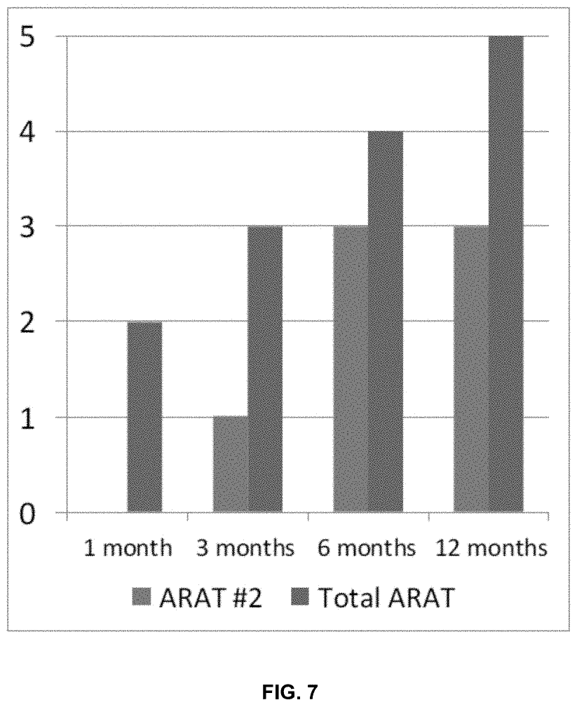

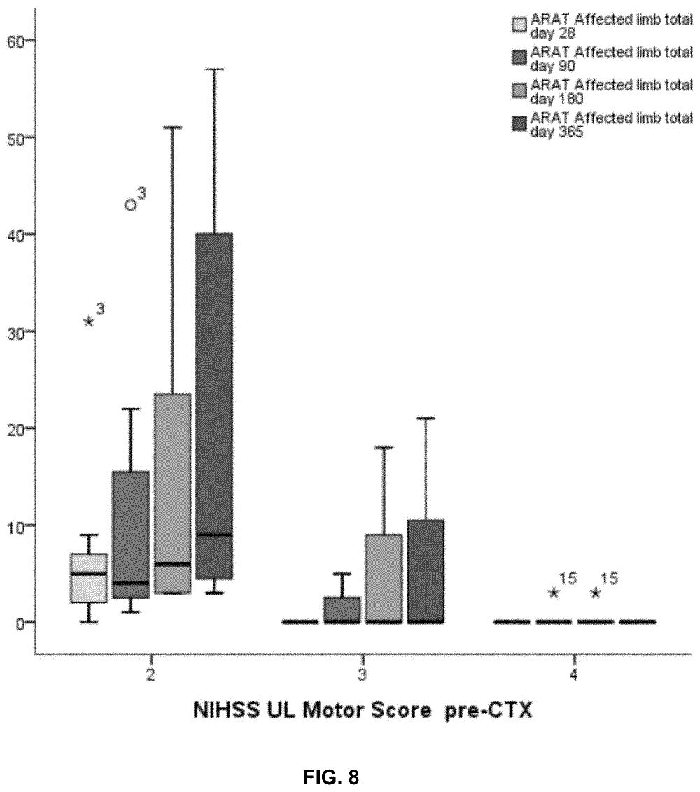

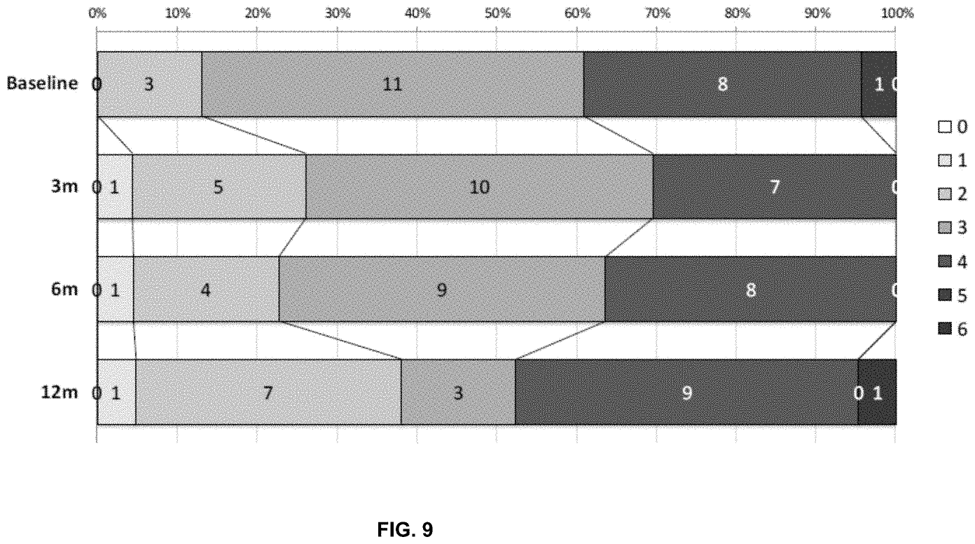

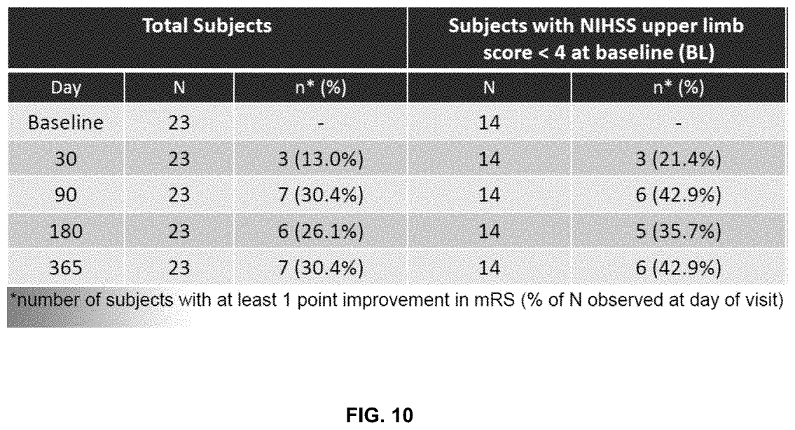

Some of the exemplary unexpected effects in stroke patients following treatment with a neural stem cell drug product and indicated in Example 2, include:
Improvements in ARAT Grasp #2, ARAT total, mRS and BI, from three months and sustained at 12 months (FIG. 6)
   Improvements as quickly as one month for mRS
No relapse back to a lower score after 12 months (FIG. 7)
An initial NIHSS Motor Arm score of 2 providing the most significant improvements on ARAT (FIG. 8)
Observation of notable benefits in patients with an initial NIHSS Motor Arm score of 3 (FIG. 8)
Improvements in the performance of patients on the modified Rankin Scale, including improvements in two different categories (FIG. 9).
Improvements in mRS for patients with an initial NIHSS Motor Arm score of 2 or 3 (FIG. 10).

Each of these observations represents an embodiment of the invention. The combination of these observations also represents an embodiment of the invention.

As shown in the Examples, some embodiments comprises administration of a single dose of 20 million CTX0E03 cells to a patient with a modified NIHSS Motor Arm Score of 2 or 3 in the paretic arm. In some embodiments, the therapeutic response comprises an increase in the total ARAT score of at least six points, and/or an improvement in mRS of at least one category, optionally two categories.

The improvement in total ARAT can in some embodiments be at least six points. This improvement can be seen, for example, within one month, within three months, within 6 months, or within 12 months. In other embodiments, the total ARAT improvement can be at least ten points (e.g., after 90 days), at least 15 points (e.g., after 180 days), or at least 30 points (e.g., after 12 months). In certain aspects, these improvements are obtained in a patient having a pre-treatment NIHSS Motor Arm Score of 2.

Neural Stem Cells

Neural stem cells are known in the art. Neural stem cells are cells with the ability to proliferate, to exhibit self-maintenance or renewal over the lifetime of the organism, and to generate clonally related neural progeny. Neural stem cells give rise to neurons, astrocytes, and oligodendrocytes during development and can replace a number of neural cells in the adult brain. Neural stem cells for use in certain aspects according to the present invention can include cells that exhibit one or more of the neural phenotypic markers Musashi-1, Nestin, NeuN, class III β-tubulin, GFAP, NF-L, NF-M, microtubule associated protein (MAP2), S100, CNPase, glypican, (especially glypican 4), neuronal pentraxin II, neuronal PAS 1, neuronal growth associated protein 43, neurite outgrowth extension protein, vimentin, Hu, internexin, 04, myelin basic protein, and pleiotrophin, among others.

In some embodiments, the neural stem cells are allogeneic.

The neural stem cell can be from a stem cell line, i.e., a culture of stably dividing stem cells. A stem cell line can to be grown in large quantities using a single, defined source. Immortalisation may arise from a spontaneous event or may be achieved by introducing exogenous genetic information into the stem cell which encodes immortalisation factors, resulting in unlimited cell growth of the stem cell under suitable culture conditions. Such exogenous genetic factors can include the gene “myc”, which encodes the transcription factor Myc. The exogenous genetic information can be introduced into the stem cell through a variety of suitable means, such as transfection or transduction. For transduction, a genetically engineered viral vehicle can be used, such as one derived from retroviruses, for example lentivirus.

Additional advantages can be gained by using a conditionally immortalised stem cell line, in which the expression of the immortalisation factor can be regulated without adversely affecting the production of therapeutically effective stem cells. This can be achieved by introducing an immortalisation factor which is inactive unless the cell is supplied with an activating agent. Such an immortalisation factor can be a gene such as c-mycER. The c-MycER gene product is a fusion protein comprising a c-Myc variant fused to the ligand-binding domain of a mutant estrogen receptor. C-MycER only drives cell proliferation in the presence of the synthetic steroid 4-hydroxytamoxifen (4-OHT) (Littlewood et al., 1995). This approach allows for controlled expansion of neural stem cells in vitro, while avoiding undesired in vivo effects on host cell proliferation (e.g., tumour formation) due to the presence of c-Myc or the gene encoding it in the neural stem cell line.

Conditionally-immortalised cell lines suitable for use with the present disclosure include, but are not limited to, the CTX0E03, STR0C05, and HPC0A07 neural stem cell lines, which have been deposited by the applicant of this patent application, ReNeuron Limited, at the European Collection of Animal Cultures (ECACC), Vaccine Research and Production laboratories, Public Health Laboratory Services, Porton Down, Salisbury, Wiltshire, SP4 0JG, with Accession No. 04091601 (CTX0E03); Accession No. 04110301 (STR0C05); and Accession No. 04092302 (HPC0A07). The derivation and provenance of these cells is described in European Patent EP1645626 B1 and U.S. Pat. No. 7,416,888.

The cells of the CTX0E03 cell line can be cultured, for example, in the following culture conditions:
Human Serum Albumin 0.03%
Transferrin, Human 5 μg/ml
Putrescine Dihydrochloride 16.2 μg/ml
Insulin Human recombinant 5 μ/ml
Progesterone 60 ng/ml
L-Glutamine 2 mM
Sodium Selenite (selenium) 40 ng/ml

Basic Fibroblast Growth Factor (10 ng/ml), epidermal growth factor (20 ng/ml), and 4-hydroxytamoxifen 100 nM can also be added to the culture conditions for cell expansion. The cells can be differentiated by removal of the 4-hydroxytamoxifen. The cells can be cultured, for example, at 5% C002/37° C. or under hypoxic conditions of 5%, 4%, 3%, 2% or 1% 02. These cell lines do not require serum to be cultured successfully. Serum is required for the successful culture of many cell lines, but contains many contaminants. A further advantage of the CTX0E03, STR0C05, or HPC0A07 neural stem cell lines, or any other cell line that does not require serum, is that the contamination by serum is avoided.

The cells of the CTX0E03 cell line are multipotent cells originally derived from 12 week human fetal cortex. The isolation, manufacture, and protocols for the CTX0E03 cell line is described in detail by Sinden, et al. (U.S. Pat. No. 7,416,888 and EP1645626 B1). The CTX0E03 cells are not “embryonic stem cells”, i.e., they are not pluripotent cells derived from the inner cell mass of a blastocyst and isolation of the original cells did not result in the destruction of an embryo. In growth medium, CTX0E03 cells are nestin-positive with a low percentage of GFAP positive cells (i.e., the population is negative for GFAP).

CTX0E03 is a clonal cell line that contains a single copy of the c-mycER transgene that was delivered by retroviral infection and is conditionally regulated by 4-OHT (4-hydroxytamoxifen). The C-mycER transgene expresses a fusion protein that stimulates cell proliferation in the presence of 4-OHT and therefore allows controlled expansion when cultured in the presence of 4-OHT. This cell line is clonal, expands rapidly in culture (with a doubling time of 50-60 hours), and has a normal human karyotype (46 XY). The cell line is genetically stable and can be grown in large numbers. The cells are safe and non-tumorigenic. In the absence of growth factors and 4-OHT, the cells undergo growth arrest and differentiate into neurons and astrocytes. Once implanted into an ischemia-damaged brain, these cells migrate only to areas of tissue damage.

The development of the CTX0E03 cell line has allowed the scale-up of a consistent product for clinical use. Production of cells from banked materials allows for the generation of cells in quantities for commercial application (Hodges et al., 2007).

The CTX0E03 drug product can be provided as a fresh (as was the case for the PISCES trial) or frozen suspension of living cells, as described in U.S. Pat. No. 9,265,795 and used in the PISCES II trial. In some embodiments, the drug product comprises CTX0E03 cells at a passage of ≤37.

In one embodiment the CTX0E03 drug product is formulated with Hypothermosol FRS (Biolife Solutions, Bothell, Wash.) as an excipient. This is suitable for intracranial administration by using stereotaxic surgical techniques. The drug product can be stored at, for example, 4° C. to 25° C. for extended periods (hours to days).

In some embodiments, the CTX clinical drug product is formulated as an “off the shelf” cryopreserved product in a solvent-free excipient (e.g. as described in U.S. Pat. No. 9,265,795) with a shelf life of many months. In certain aspects, this formulation comprises Trolox (6-hydroxy-2,5,7,8-tetramethylchroman-2-carboxylic acid), Na⁺, K⁺, Ca²⁺, Mg²⁺, Cl⁻, H₂PO₄⁻, HEPES, lactobionate, sucrose, mannitol, glucose, dextran-40, adenosine, and glutathione. One or more, for example two, three, or four, of these excipients can optionally be removed or replaced. In some embodiments, the formulation does not comprise a dipolar aprotic solvent, such as DMSO.

Clinical release criteria for neural stem cell products can include measures of sterility, purity (e.g., cell number, cell viability), and a number of other tests of identity, stability, and potency that are required for clinical product release or for information, as requested by regulatory authorities. The tests employed for CTX0E03 are summarised in Table 1, below.

TABLE 1
Identity, Stability, and Potency Tests That Are Employed to Characterize
CTX Cell Banks and/or Drug Products (for Phase II Trial)

| Test | Outcome |
|---|---|
| PCR Sequencing of cDNA | Sequence of insert conforms to transgene |
| | identity. No insertions, deletions, or |
| | mutations from expected sequence |
| Determination of Flanking Nucleotide | Consistent with published sequence |
| Sequence | |
| PCR across integration site | PCR across integration site confirms cell line |
| | identity |
| Karyology | Comparable with published normal |
| | chromosome, male XY |
| Viability and growth | ≥70% viability on recovery. Viable cell |
| | numbers at least double within 7 days |
| c-mycER^{TAM} gene copy number | Modal ~1 (range 0.87-3.46) |
| (PCR) | |
| Phenotypic marker (Nestin) | At least 95% of cells are Nestin positive |
| Position, sequence, and indication of | Chromosomal (Chr 13) localization of |
| number of integrated target gene by | integrated c-mycER^{TAM} sequences |
| fluorescent in situ hybridization | |
| Potency | Cell dose-dependent IL-10 production in co- |
| | culture with U937 monocyte cell line |
| Neural differentiation | Upregulation of Tub-β3, GFAP, and GAL-C |
| | marker expression by qPCR after seeding |
| | into ALVATEX ® three-dimensional cell matrix |

In some embodiments, the neural stem cells are administered in the undifferentiated state.

The CTX0E03 cell line has been previously demonstrated, using a human PBMC assay, not to be immunogenic. The lack of immunogenicity allows the cells to avoid clearance by the host/patient immune system and thereby to exert their therapeutic effect without a deleterious immune and inflammatory response.

Pollock et al., 2006 describe that transplantation of CTX0E03 in a rat model of stroke (MCAo) caused statistically significant improvements in both sensorimotor function and gross motor asymmetry at 6-12 weeks post-grafting. These data indicate that CTX0E03 has the appropriate biological and manufacturing characteristics necessary for development as a therapeutic cell line.

Stevanato et al., 2009 confirms that CTX0E03 cells down-regulated c-mycERTAM transgene expression both in vitro following EGF, bFGF, and 4-OHT withdrawal and in vivo following implantation in MCAo rat brain. The silencing of the c-mycERTAM transgene in vivo provides an additional safety feature of CTX0E03 cells for potential clinical application.

Smith et al., 2012 describe preclinical efficacy testing of CTX0E03 in a rat model of stroke (transient middle cerebral artery occlusion). The results indicate that CTX0E03 implants robustly recover behavioural dysfunction over a 3 month time frame and that this effect is specific to their site of implantation. Lesion topology is potentially an important factor in the recovery, with a stroke confined to the striatum showing a better outcome compared to a larger area of damage.

Neural retinal stem cell lines (for example as described in U.S. Pat. No. 7,514,259) can also be used according to the invention.

Neural stem cells for use in the methods according to the invention can also be fetal, embryonic, or adult neural stem cells, such as has been described in U.S. Pat. Nos. 5,851,832; 6,777,233; 6,468,794; 5,753,506 and International Patent Application Publication No. WO2005/121318. The fetal tissue can be human fetal cortex tissue. The cells can be selected as neural stem cells from the differentiation of induced pluripotent stem (iPS) cells, as has been described by Yuan et al., 2011 or a directly induced neural stem cell produced from somatic cells such as fibroblasts (for example by constitutively inducing Sox2, Klf4, and c-Myc while strictly limiting Oct4 activity to the initial phase of reprogramming as recently disclosed by Their et al., 2012). Human embryonic stem cells can be obtained by methods that preserve the viability of the donor embryo, as is known in the art (e.g., Chung et al., 2008). Such non-destructive methods of obtaining human embryonic stem cell can be used to provide embryonic stem cells from which neural stem cells can be obtained. Alternatively, stem cells of the invention can be obtained, for example, from adult stem cells, iPS cells, or directly-induced neural stem cells. Accordingly, stem cells of the disclosure can be produced by multiple methods that do not require the destruction of a human embryo or the use of a human embryo as a base material.

In some embodiments, neural stem cells for use in the methods of the invention are cells of a different type that have been modified to be recognisable as neural stem cells. In some embodiments, other stem cell types are modified to express one or more markers of stem cells. In certain embodiments, the neural stem cell is derived from a mesenchymal stem cell (MSC). For example, International Patent Application Publication No. WO2005/100552, which is incorporated herein by reference in its entirety, describes a method of producing cells exhibiting neuronal progenitor (stem) cell characteristics from material comprising marrow adherent stem cells (MASC). See also, Dezawa et al., 2004 J Clin. Invest. 113: 1701-1710, which is also incorporated herein by reference in its entirety.

In some embodiments, neural stem cells derived from another cell type (for example, an MSC such as a Marrow Adherent Stem Cell) are mitotic and express nestin and optionally other cell markers specific for neural precursor/neural progenitor cells. Such MSC-derived neural cells can differentiate into neurons, glia, and oligodendrocytes, and precursors of any of the foregoing. NSCs can be derived from MASCs according to methods disclosed in WO2005/100552. Marrow adherent stem cells (MASCs) can be defined as being stem cells that are conventionally recognized as differentiating into several types of cells found primarily in connective tissues, including but not limited to, osteoblasts, adipocytes, chondrocytes, and myocytes. In an embodiment, human MASCs express CD29, and CD90, but are negative for CD15, CD34, CD11b/c, CD31, CD45 and von Willebrand Factor.

Methods of producing NSCs from other cells such as MSCs can include regulating cellular pathways in the stem cells that are associated with glial transdifferentiation; wherein the cellular pathways are sufficiently regulated to induce at least a portion of the stem cells to transdifferentiate into cells exhibiting neuronal progenitor cell characteristics; and optionally with the proviso that the regulating does not comprise transfection of the stem cells with notch intracellular domain.

Another method for producing NSCs from other cells such as MSCs can include incubating stem cells (e.g., marrow adherent stem cells) with a glial regulating agent in an amount sufficient to induce at least a portion of the stem cells to transdifferentiate into cells exhibiting neuronal progenitor cell characteristics; optionally with the proviso that the interacting does not comprise transfection of the marrow adherent stem cells with notch intracellular domain.

MASCs can be cultured in the presence of glial regulating agents with the intent that the glial regulating agents either interact with MASC cell surface receptors or are transported into the interior of the MASCs to interact with internal cellular pathways. Such transportation can be passive, such as diffusive transport, or active, such as through active transporters, or a mixture of the two. In vitro incubations can be performed in a conventional manner, for instance by incubating cultures of MASCs in alpha-MEM, or similar media, to which glial regulating agent(s) are added.

Glial regulating agents are substances that possess the characteristic of inhibiting transdifferentiation of MASCs into glial cells and promoting their transdifferentiation into neural stem cells. Glial regulating agents can act through a variety of different mechanisms to direct MASCs away from the glial fate. For instance, pro-neural basic helix-loop-helix transcription factors such as Mash 1, Math 1, and neurogenin 1 are believed to be activators of neuronal gene expression. Proneural genes are believed to drive neuronal transdifferentiation of MASCs while inhibiting glial transdifferentiation. One mechanism by which glial transdifferentiation can be inhibited is through the regulation of STAT-mediated signal transduction. Signal transduction by STAT is believed to be triggered by phosphorylation which is believed to be catalyzed by the Janus family of tyrosine kinases (JAK). Inhibition of the JAK-STAT signal transduction therefore can regulate glial transdifferentiation pathways and promote the neuronal fate of MASCs. JAK/STAT inhibitors can include inhibitors of STAT1 and STAT3. In certain embodiments, such JAK/STAT inhibitors comprise RNAi for gene silencing of the JAK/STAT pathway, antisense oligonucleotides to down-regulate the JAK/STAT pathway, or the small molecule JAK inhibitor 4-(4′-hydroxyphenyl)amino-6,7-dimethoxyquinazoline. In other embodiments, glial regulating agents include antagonists of BMP2 or 7 (bone morphogenic protein). Such antagonists can comprise whole or portions of gene products from genes expressing Noggin, Chordin, Follistatin, sonic hedgehog (SHH), or agonists of these genes. Glial regulating agents include, but are not limited to, Hes inhibitors, including but not limited to Hes 1 and/or Hes 5 inhibitors. In certain embodiments, such Hes inhibitors comprise RNAi for gene silencing of Hes, or antisense oligonucleotides to down-regulate Hes. Glial regulating agents can include, but are not limited to, inhibitors of Id-1 (See S. Tzeng et al., Id1, Id2, and Id3 gene expression in neural cells during development. Glia. 1998 December; 24(4):372-81). In certain embodiments, such Id-1 inhibitors comprise RNAi for gene silencing of Id-1, or antisense oligonucleotides to down-regulate Id-1.

Glial regulating agents can also include, but are not limited to, inhibitors of mammalian homologs of Drosophila glide/gem (glial cells missing), including but not limited to Gcm1 (murine) or GCMB (human). Glial regulating agents can also include, but are not limited to, inhibitors of Sox9, which can be a transcription factor for oligodendrocyte lineage. In certain embodiments, such Sox9 inhibitors comprise RNAi for gene silencing of Sox9, or antisense oligonucleotides to down-regulate Sox9.

In other embodiments, glial regulating agents include, but are not limited to, inhibitors of Neurogenin3, inhibitors of ciliary neurotrophic factor (CNTF), whole or portions of gene products from genes expressing Wnt1 (which strongly inhibits gliogenesis), or whole or portions of gene products from genes expressing a subset of neural basic helix-loop-helix (bHLH) factors that play instructive roles during neurogenesis or are expressed in proliferating CPCs.

In one embodiment, human MSC-derived NSCs are EfnB2⁺, CD90⁻, and PDGF receptor beta⁻. One or more of these markers can be used to separate the NSCs from MASCs using FACS following glial transdifferentiation of the MASCs, for example as described in WO2005/100552.

Cells descended from marrow adherent stem cells (MASCs) that have been engineered to express an exogenous Notch intracellular domain (NICD) are known in the art. One example is the cells known as “SB623 cells”, as described for example in International Patent Application Publication No. WO2014/058464 (incorporated herein by reference). The production of such cells can include contacting a culture of MASCs with a polynucleotide comprising sequences encoding a NICD (e.g., by transfection), followed by enrichment of transfected cells by drug selection and further culture. See, for example, U.S. Pat. No. 7,682,825 (issued Mar. 23, 2010); U.S. Patent Application Publication No. 2010/0266554 (Oct. 21, 2010); and International Patent Application Publication WO2009/023251 (Feb. 19, 2009); all of which are incorporated herein by reference, in their entireties, for all purposes, including describing isolation of mesenchymal stem cells and conversion of mesenchymal stem cells to SB623 cells (denoted “neural precursor cells” and “neural regenerating cells” in those documents).

Additional details on the preparation of SB623 cells, and methods for making cells with properties similar to those of SB623 cells which can be used in the methods disclosed herein, are found in U.S. Pat. No. 7,682,825; and U.S. Patent Application Publication Nos. 2010/0266554 and 2011/0229442; the disclosures of which are incorporated by reference herein in their entireties for all purposes, including providing additional details on the preparation of SB623 cells, and for providing methods for making cells with properties similar to those of SB623 cells. See also, Dezawa et al., 2004 J Clin. Invest. 113: 1701-1710.

MSC-derived NSCs are being used in a clinical trial (NCT02448641) in Patients With Chronic Motor Deficit From Ischemic Stroke. The NCT02448641 trial uses the SB623 cells noted above. Steinberg et al., (Stroke. 2016 July; 47(7):1817-24) also describes these cells.

Suitable methods of handling the MSC-derived NSCs are known, including those methods disclosed, for example, in published United States Patent Application No. 2002/0012903 to Goldman et al.

In some embodiments, the stem cells of the disclosure are isolated. The term “isolated” indicates that the cell or cell population to which it refers is not within its natural environment. The isolated cell or cell population has been, for example, substantially separated from surrounding tissue. In some embodiments, the cell or cell population is substantially separated from surrounding tissue if the sample contains at least about 75%, in some embodiments at least about 85%, in some embodiments at least about 90%, and in some embodiments at least about 95% stem cells. In other words, the sample is substantially separated from the surrounding tissue in some embodiments if the sample contains less than about 25%, in some embodiments less than about 15%, and in some embodiments less than about 5% of materials other than the stem cells. Such percentage values refer to percentage by weight. The term encompasses cells which have been removed from the organism from which they originated, and exist in culture. The term also encompasses cells which have been removed from the organism from which they originated, and subsequently re-inserted into an organism. The organism which contains the re-inserted cells may be the same organism from which the cells were removed, or it may be a different organism.

Cell Populations

The invention provides a population of isolated neural stem cells, wherein the population essentially comprises only stem cells of the invention, i.e., the stem cell population is substantially pure. In some aspects, the stem cell population comprises at least about 75%, or at least 80% (in other aspects at least 85%, 90%, 91%, 92%, 93%, 94%, 95%, 96%, 97%, 98%, 99%, 99.5%, 99.9% or 100%) of the stem cells of the disclosure, with respect to other cells that make up a total cell population. For example, with respect to neural stem cell populations, this term means that in some embodiments there are at least about 75%, in some embodiments at least about 85%, in some embodiments at least about 90%, and in some embodiments at least about 95% pure, neural stem cells compared to other cells that make up a total cell population. The term “substantially pure” therefore refers to a population of stem cells of the present disclosure that in some embodiments contains fewer than about 25%, in some embodiments fewer than about 15%, and in some embodiments fewer than about 5%, of cells that are not neural stem cells.

The isolated neural stem cells of the disclosure can be characterised by a distinctive expression profile for certain markers and are distinguished from stem cells of other cell types. When a marker is described herein, its presence or absence can in some embodiments be used to distinguish the neural stem cell.

In one embodiment, the invention relates to a neural stem cell population characterised in that the cells of the population express one, two, three, four, five or more, for example all, of the markers Nestin, Sox2, GFAP, βIII tubulin, DCX, GALC, TUBB3, GDNF, and IDO.

In some embodiments, the neural stem cells are nestin positive.

A “marker” refers to a biological molecule whose presence, concentration, activity, or phosphorylation state can be detected and used to identify the phenotype of a cell.

In some embodiments, a neural stem cell population of the disclosure is considered to carry a marker if at least about 70% of the cells of the population show a detectable level of the marker. In other aspects, at least about 80%, at least about 90%, or at least about 95%, or at least about 97%, or at least about 98% or more of the population show a detectable level of the marker. In certain aspects, at least about 99% or 100% of the population show detectable level of the markers. Quantification of the marker can be detected, for example, through the use of a quantitative RT-PCR (qRT-PCR) or through fluorescence activated cell sorting (FACS). It should be appreciated that this list is provided by way of example only, and is not intended to be limiting. In some embodiments, a neural stem cell of the disclosure is considered to carry a marker if at least about 90% of the cells of the population show a detectable level of the marker as detected by FACS.

The term “expressed” is used to describe the presence of a marker within a cell. In order to be considered as being expressed, a marker must be present at a detectable level. By “detectable level,” it is meant that the marker can be detected using one of the standard laboratory methodologies such as qRT-PCR, or qPCR, blotting, Mass Spectrometry or FACS analysis. A gene is considered to be expressed by a cell of the population of the disclosure if expression can be reasonably detected at crossing point (cp) values below or equal to 35 (standard cut off on a qRT-PCR array). The cp represents the point where the amplification curve crosses the detection threshold, and can also be reported as crossing threshold (ct).

The terms “express” and “expression” have corresponding meanings. At an expression level below this cp value, a marker is considered not to be expressed. The comparison between the expression level of a marker in a stem cell of the disclosure, and the expression level of the same marker in another cell, such as for example a mesenchymal stem cell, can preferably be conducted by comparing the two cell types that have been isolated from the same species. Preferably this species is a mammal, and more preferably this species is human. Such comparison can conveniently be conducted using, for example, a reverse transcriptase polymerase chain reaction (RT-PCR) experiment.

As used herein, the term “significant expression” or its equivalent terms “positive” and “+” when used in regard to a marker shall be taken to mean that, in a cell population, more than 20%, preferably more than, 30%, 40%, 50%, 60%, 70%, 80%, 90%, 95%, 98%, 99%, or even all of the cells express said marker.

As used herein, “negative” or “−” as used with respect to markers shall be taken to mean that, in a cell population, fewer than 20%, 10%, preferably fewer than 9%, 8%, 7%, 6%, 5%, 4%, 3%, 2%, 1%, or none of the cells express said marker.

Expression of cell surface markers can be determined, for example, by means of flow cytometry and/or Fluorescence activated cell sorting (FACS) for a specific cell surface marker using conventional methods and apparatus (for example a Beckman Coulter Epics XL FACS system used with commercially available antibodies and standard protocols known in the art) to determine whether the signal for a specific cell surface marker is greater than a background signal. The background signal is defined as the signal intensity generated by a non-specific antibody of the same isotype as the specific antibody used to detect each surface marker. For a marker to be considered positive, the specific signal observed can be, for example, more than 20%, preferably stronger than 30%, 40%, 50%, 60%, 70%, 80%, 90%, 100%, 500%, 1000%, 5000%, 10000% or above, greater relative to the background signal intensity. Alternative methods for analysing expression of cell surface markers of interest include visual analysis by electron microscopy using antibodies against cell-surface markers of interest.

Stem Cell Culture and Production

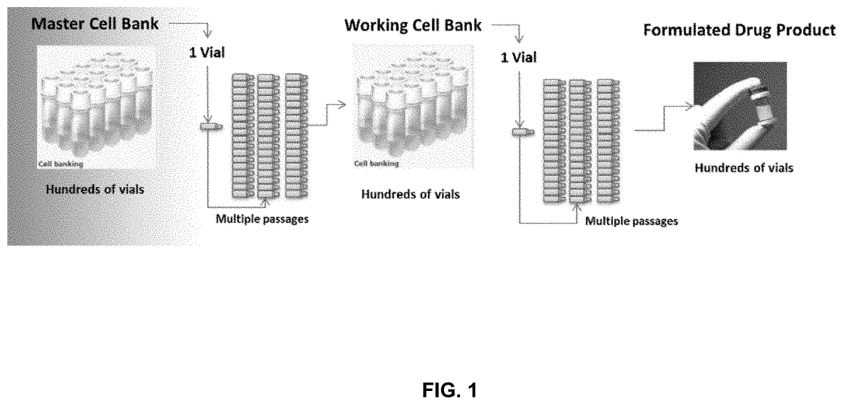

In some embodiments, stem cells intended for use in therapy are banked and a drug product is made from the banked cells according to Good Manufacturing Processes (GMP). A master cell bank can comprise hundreds of vials, each of which can in turn be passaged multiple times to produce a working cell bank comprising hundreds of vials, each of which can be passaged multiple times to produce hundreds of Drug Product vials. In this way, the drug product (DP) can be prepared for each patient at the required dose. An exemplary production scheme is shown in FIG. 1.

Simple bioreactors for stem cell culture include, for example, single compartment flasks, such as the commonly-used T-175 flask (e.g. the BD Falcon™ 175 cm² Cell Culture Flask, 750 ml, tissue-culture treated polystyrene, straight neck, blue plug-seal screw cap, BD product code 353028).

In some embodiments, the cells for therapy are taken from proliferating neural stem cells cultured in T-175 or T-500 flasks.

In some embodiments, the CTX0E03 drug product comprises CTX0E03 cells at a passage of ≤37.

Bioreactors can also have multiple compartments, as is known in the art. These multi-compartment bioreactors can contain at least two compartments separated by one or more membranes or barriers that separate the compartment containing the cells from one or more compartments containing gas and/or culture medium. Multi-compartment bioreactors are well-known in the art. An example of a multi-compartment bioreactor is the Integra CeLLine bioreactor, which contains a medium compartment and a cell compartment separated by means of a 10-kDa semi-permeable membrane. This membrane can allow a continuous diffusion of nutrients into the cell compartment with a concurrent removal of an inhibitory waste product. The individual accessibility of the compartments can allow the cells to be supplied with fresh medium without mechanically interfering with the culture. A silicone membrane forms the cell compartment base and can provide an optimal oxygen supply and control of carbon dioxide levels by providing a short diffusion pathway to the cell compartment. Any other multi-compartment bioreactor can also be used according to the disclosure.

The term “culture medium” or “medium” is recognized in the art, and refers generally to any substance or preparation used for the cultivation of living cells. The term “medium”, as used in reference to a cell culture, includes the components of the environment surrounding the cells. Media can be solid, liquid, gaseous, or a mixture of phases and materials. Media include liquid growth media as well as liquid media that do not sustain cell growth. Media also include gelatinous media such as agar, agarose, gelatin, and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to a medium that is made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an optical density OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium can be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

Pharmaceutical Compositions

The neural stem cells of the disclosure are useful in therapy and can therefore be formulated as a pharmaceutical composition. A pharmaceutically acceptable composition can include at least one pharmaceutically acceptable carrier, diluent, vehicle and/or excipient in addition to the neural stem cells of the disclosure. An example of a suitable carrier is Ringer's Lactate solution. A thorough discussion of such components is provided in Gennaro, 2000, Remington: The Science and Practice of Pharmacy, 20th edition, ISBN: 0683306472.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition, if desired, can also contain minor amounts of pH buffering agents. The composition can comprise storage media such as HYPOTHERMOSOL®, commercially available from BioLife Solutions Inc., USA. Examples of suitable pharmaceutical carriers are described in “Remington's Pharmaceutical Sciences” by E. W. Martin. Such compositions can contain a prophylactically or therapeutically effective amount of a prophylactic or therapeutic stem cell in purified form, together with a suitable amount of carrier so as to provide the form for proper administration to the subject. The formulation can be selected to suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention can be in a variety of forms. These include, for example, semi-solid, and liquid dosage forms, such as lyophilized preparations, frozen preparations, liquid solutions or suspensions, and injectable and infusible solutions. In some embodiments, the pharmaceutical composition is injectable.

Pharmaceutical compositions can be in aqueous form. Compositions can include a preservative and/or an antioxidant.

To control tonicity, the pharmaceutical composition can comprise a physiological salt, such as a sodium salt. Sodium chloride (NaCl) is preferred, which can be present at between 1 and 20 mg/ml. Other salts that can be present include potassium chloride, potassium dihydrogen phosphate, disodium phosphate dehydrate, magnesium chloride, calcium chloride, and combinations thereof.

Compositions can include one or more buffers. Suitable buffers include, but are not limited to, a phosphate buffer, a Tris buffer, a borate buffer, a succinate buffer, a histidine buffer, or a citrate buffer. Buffers can be included at a concentration in, for example, the 5-20 mM range. The pH of a composition can be between 5 and 8, e.g, between 6 and 8, between 6.5 and 7.5, or between 7.0 and 7.8.

In some embodiments, the composition is sterile. In some embodiments, the composition is non-pyrogenic.

In some embodiments, the cells are suspended in a composition comprising 1, 2, 3, 4, 5, 6, 7, 8, 9, 10, or more excipients selected from 6-hydroxy-2,5,7,8-tetramethylchroman-2-carboxylic acid (TROLOX®), Na⁺, K⁺, Ca²⁺, Mg²⁺, Cl⁻, H₂P0₄⁻, HEPES, lactobionate, sucrose, mannitol, glucose, dextron-40, adenosine, and glutathione. In one embodiment the composition comprises all of these excipients. In some embodiments, the composition does not include a dipolar aprotic solvent, e.g., DMSO. Suitable compositions are available commercially, e.g. HYPOTHERMASOL®-FRS. Such compositions are advantageous as they allow the cells to be stored at 4° C. to 25° C. for extended periods (hours to days) or preserved at cryothermic temperatures, e.g., temperatures below −20° C. The stem cells can then be administered in this composition after thawing.

In some embodiments, the composition comprises between 4×10⁴ and 7×10⁴ viable cells/μL, e.g., 5×10⁴ viable cells/μL.

Treatment of Stroke Patients

The neural stem cells of the invention are useful in the treatment of stroke, e.g., ischemic stroke. Accordingly, the disclosure includes a method of treating ischemic stroke in a patient using neural stem cells. In some embodiments, the ischemic stroke is a supratentorial ischemic stroke.

The term “patient” includes human and other mammalian subjects that receive either therapeutic treatment as set out herein. In some embodiments, the patient is a human.

The CTX0E03 cell line is currently being tested in a clinical trial for treatment of disabled stroke patients (Clinicaltrials.gov Identifier: NCT01151124). WO2012/004611 describes the use of the CTX0E03 cells in treating psychiatric disorders including unipolar and bipolar depression, schizophrenia, obsessive compulsive disorder, autism, and autistic syndrome disorders.

As used herein, the terms “treat,” “treatment,” “treating,” and “therapy,” when used directly in reference to a patient or subject, shall be taken to mean the amelioration of one or more symptoms associated with a disorder, or the prevention or prophylaxis of a disorder or one or more symptoms associated with a disorder. The disorder to be treated can be ischemic stroke. Amelioration or prevention of symptoms results from the administration of the neural stem cells of the invention, or of a pharmaceutical composition comprising these cells, to a subject in need of said treatment.

The PISCES II trial, set out in the Examples, aimed to demonstrate the effect of CTX0E03 cells on improving the outcome of patients during the rehabilitation phase following an ischemic stroke, and to provide further safety data in a larger group of patients. The inclusion criteria for the trial can be summarised as:
Male and Female Patients
ARAT Test #2 of 0 or 1
NIHSS Upper limb motor score of 4, 3, or 2

Such patients, with stable marked paresis of the affected arm (inability to lift the arm off of a table against gravity) have a probability of spontaneous improvement of the arm (sufficient to use it for feeding) less than 5% in the absence of the cell lines and treatments provided herein.

The study procedures can be summarised as:
CTX 20 million cells injected into brain (putamen) on the affected side
Follow up for 12 months
Physiotherapy minimum 1.5 hours/week for 6 weeks
Primary Measure: ARAT test #2, 2 point improvement
Secondary Measures: ARAT, Fugl-Meyer, NIHSS, mRS, Barthel Index.

The PISCES II trial was modified from an initial Simon two-stage design with the primary endpoint at Day 180 to a single cohort design with the primary endpoint at Day 90, and the target population was expanded from patients with a baseline NIHSS arm score of 2 or 3 to also include those with a score of 4.

NIHSS Upper Limb Motor Score

The National Institutes of Health Stroke Scale, or NIH Stroke Scale (NIHSS) is a tool used by healthcare providers to quantify objectively the impairment caused by a stroke. The NIHSS is composed of 11 items, each of which scores a specific ability between a 0 and 4. For each item, a score of 0 typically indicates normal function in that specific ability, while a higher score is indicative of some level of impairment. The individual scores from each item are added together to calculate a patient's total NIHSS score. The maximum possible score is 42, with the minimum score being a 0.

A Modified NIHSS is described by Meyer et al., Stroke. 2002; 33:1261-1266, which removed four questions from the NIHSS. The maximum possible score with the use of this simplified scale is 31, compared with 42 for the original scale.

One of the items in both the NIHSS and modified NIHSS is the “Motor Arm” test. With palm facing downwards, the patient extends one arm 90 degrees out in front if the patient is sitting, and 45 degrees out in front if the patient is lying down. If necessary, the patient is helped into the correct position. As soon as the patient's arm is in position the investigator should begin verbally counting down from 10 while simultaneously counting down on his or her fingers in full view of the patient. The investigator observes to detect any downward arm drift prior to the end of the 10 seconds. Downward movement that occurs directly after the investigator places the patient's arm in position should not be considered downward drift. The test is repeated for the opposite arm. This item should be scored for the right and left arm individually. The scores are as follows:
0—No arm drift; the arm remains in the initial position for the full 10 seconds
1—Drift; the arm drifts to an intermediate position prior to the end of the full 10 seconds, but not at any point relies on a support
2—Limited effort against gravity; the arm is able to obtain the starting position, but drifts down from the initial position to a physical support prior to the end of the 10 seconds
3—No effort against gravity; the arm falls immediately after being helped to the initial position, however the patient is able to move the arm in some form (e.g. shoulder shrug)
4—No movement; patient has no ability to enact voluntary movement in this arm

The PISCES II study tested patients with an NIHSS Motor Arm Score of 2, 3, or 4 at baseline (i.e. before treatment). Surprising results were observed in patients with an NIHSS Upper Arm Score of 2 or 3. Accordingly, the disclosure relates in some embodiments to the subset of patients having an NIHSS Upper Arm Score of 2 or 3 prior to treatment.

ARAT Test and ARAT Test (Grasp) #2

The ARAT test #2 (also known as the ARAT Grasp test #2) is the second of the tests within the Grasp subscale of the Action Research Arm Test (ARAT). The ARAT is a well-known 19-item observational measure used by physical therapists and other health care professionals to assess upper extremity performance (coordination, dexterity and functioning) in stroke recovery, brain injury, and multiple sclerosis populations. The ARAT was originally described by Lyle in 1981 as a modified version of the Upper Extremity Function Test and was used to examine upper limb functional recovery post damage to the cortex (International Journal of Rehabilitation Research. (1981); 4(4), 483-492). Items comprising the ARAT are categorized into four subscales (grasp, grip, pinch, and gross movement) and arranged in order of decreasing difficulty, with the most difficult task examined first, followed by the least difficult task.

The ARAT test #2 (grasp) tests the placement of a 2.54-cm³ block, from the surface of a table to a shelf located 37 cm above the starting point.

Each of the 19 items comprising the ARAT is scored using a 4 point ordinal scale, as follows:
0—no movement
1—movement task is partially performed
2—movement task is completed but takes abnormally long
3—movement is performed normally.

For the Grasp Test #2, these are typically assessed as:
0—no part of task completed
1—some effort, but does not perform task
2—performs within 5-60 seconds
3—performs within 5 seconds.

Scores on the Total ARAT (19 items in total) therefore range from 0-57 points, with a maximum score of 57 points indicating better performance. There are no cut off scores because this assessment is continuous and based on a subject's observed mobility.

The ARAT can be used to predict the functional recovery of the upper extremity in stroke rehabilitation. Scores of less than 10 points, between 10-56 points, and 57 points correlate with poor, moderate, and good recovery respectively.

In some embodiments, the patient to be treated as disclosed herein has a Score of 0 or 1 for test #2 of the ARAT Grasp test #2.

The Primary Measure in the PISCES II trial is two Responders in the ARAT grasp test #2 at 3 months post-treatment, wherein a Responder is a patient showing a two-point improvement at six-months post-treatment.

One of the secondary measures in the PISCES II trial is to assess the efficacy of intracranial CTX DP in restoring upper limb function following an ischemic stroke using the Total ARAT, over a 12-month Time Frame.

As shown in Example 2, the trial revealed that Neural Stem Cell treatment of the stroke patients led to at least a 2-point improvement in the ARAT Subtest #2 (grasp) in one patient after three months and in three patients after 6 months and 12 months. Furthermore, at least a six point improvement was observed in Total ARAT in two patients after just one month, 3 patients at 3 months, 4 at six months, and 5 at 12 months.

Furthermore, FIG. 7 demonstrates that the 3 responders with at least 2-point improvement observed for ARAT Test #2 (grasp), responded at 3, 6, and 12 months and did not relapse back to a lower score. Similarly, FIG. 7 shows that for Total ARAT the at least 6 point improvement is observed in five responders, at 1, 1, 3, 6, and 12 months.

Even more surprising results are shown in FIG. 8. These data reveal that the improvements in ARAT are observed in patients with residual movement of the affected arm (an NIHSS Motor Arm score of 2 or 3) at baseline. However, no improvement is seen in patients with no movement in the affected arm (NIHSS Motor Arm Score of 4).

The largest and most surprising benefit is seen in patients with an NIHSS score of 2 before treatment, where a significant improvement in Total ARAT response is observed.

Modified Rankin Scale

The modified Rankin Scale (mRS) is a commonly used scale for measuring the degree of disability or dependence in the daily activities of people who have suffered a stroke or other causes of neurological disability. The scale runs from 0-6, running from perfect health without symptoms to death.
0—No symptoms.
1—No significant disability. Able to carry out all usual activities, despite some symptoms.
2—Slight disability. Able to look after own affairs without assistance, but unable to carry out all previous activities.
3—Moderate disability. Requires some help, but able to walk unassisted.
4—Moderately severe disability. Unable to attend to own bodily needs without assistance, and unable to walk unassisted.
5—Severe disability. Requires constant nursing care and attention, bedridden, incontinent.
6—Dead.

The Rankin Focussed Assessment version of the modified Rankin Scale (RFA-mRS) is described by Saver et al., Stroke. 2010 May; 41(5): 992-995, which explains that the assessment was developed by selecting and refining elements from prior instruments. The RFA takes 3-5 minutes to apply and provides clear, operationalized criteria to distinguish the 7 assignable global disability levels.

Example 2 reports that Neural Stem Cell treatment of the stroke patients led to at least one category of improvement in the mRS in three patients after one month, and in seven patients after 3 months.

Furthermore, FIG. 9 demonstrates the distribution of the mRS results following neural stem cell therapy. These results indicate an improvement at 12 months (or final measured) compared to baseline in seven patients. Six patients showed an improvement in one category. One patient showed a striking improvement by two categories.

Accordingly, in one embodiment of the invention, the administration of Neural Stem Cells to the brain of a stroke patient results in an improvement of at least one category on the mRS after 12 months or less. In another embodiment, the administration of Neural Stem Cells to the brain of a stroke patient results in an improvement by two categories on the mRS. In some embodiments, these improvements are achieved following a single administration of 20 million cells. Exemplary cells are CTX0E03 cells.

FIG. 10 shows that improvements in mRS is greatest in patients with residual movement of the affected arm (NIHSS Motor Arm Score of 2 or 3) at baseline. This patient subgroup seeing the greatest benefit is consistent with the results observed for ARAT.

In one embodiment, patients have an mRS pre-treatment of 3 or 4.

In a further embodiment, the patient is assessed for mRS at six-months post-treatment.

Barthel Index

The Barthel Index (BI) consists of 10 items that measure a person's daily functioning, particularly the activities of daily living (ADL) and mobility. The items include feeding, transfers from bed to wheelchair and to and from a toilet, grooming, walking on a level surface, going up and down stairs, dressing, and continence of bowels and bladder. Each performance item is rated on this scale with a given number of points assigned to each level or ranking. The amount of time and physical assistance required to perform each item are used in determining the assigned value of each item. External factors within the environment affect the score of each item. The BI can be used to determine a baseline level of functioning and can be used to monitor improvements in activities of daily living over time.

The ten variables addressed in the Barthel scale are: (i) presence or absence of fecal incontinence; (ii) presence or absence of urinary incontinence; (iii) help needed with grooming; (iv) help needed with toilet use; (v); help needed with feeding; (vi) help needed with transfers (e.g., from chair to bed); (vii) help needed with walking; (viii) help needed with dressing; (ix) help needed with climbing stairs; and (x) help needed with bathing.

The BI was developed by Mahoney and Barthel in 1965 and is now widely used in rehabilitation. 10 activities are scored, and the values are added to give a total score from 0 (totally dependent) to 100 (completely independent).

A modified version of the BI has been introduced (Colin et al., 1988). The modified scale gives a maximum score of 20, with scores ranging from 0 to 2 or 3 for each activity.

The BI can be derived from the UK FIM+/−FAM, and NPDS/NPDS-H by means of a computerised algorithm within the UKROC software (see, for example, Nyein et al., Clinical Rehabilitation 1999; 13: 56-63).

Example 2 shows that Neural Stem Cell treatment of the stroke patients led to an improvement of at least 9 points in the BI in six patients within one month, and in eight patients within three months.

Fugl-Meyer Assessment (FMA)

The FMA assessment was introduced to PISCES II in a Protocol Amendment (#8) and so is available for a subset of patients. It comprises motor assessments for upper extremities (33 tests), lower extremities (17 tests), and sensory assessments (12 tests), giving a total motor and sensory score of 0-124, where higher numbers correspond to a better medical outcome.

The FMA total motor and sensory score was observed, as described in the Examples, to be improved by at least 5 points by Day 90 and to continue to improve, reaching a change of 8 at Day 365.

Dosing

The neural stem cells can be administered at a dose and schedule sufficient to provide the therapeutic effect. This may be referred to as an “effective amount”. In some embodiments, the dose will involve a single dose. The, or each, dose can comprise, for example, at least 1 million cells, at least 2 million cells, or at least 5 million cells, for example 10 million cells or more. As shown in the Examples, an exemplary single dose is between 16×10⁶ and 28×10⁶ cells, for example the single dose can comprise around 20 million cells.

The pharmaceutical composition can be administered by any appropriate route, which will be apparent to the skilled person depending on the disease or condition to be treated and taking due note of the guidance provided in the Examples. Available routes of administration for pharmaceuticals include, for example, intravenous, intra-arterial, intramuscular, subcutaneous, intracranial, intranasal or intraperitoneal. For treatment of a disorder of the brain such as stroke, one option is to administer the stem cells intra-cerebrally, close to (e.g. not at the same location as) or at the site of damage or disease.

The neural stem cells will be administered at a therapeutically or prophylactically-effective dose, which will be apparent to the skilled person. Due to the low or non-existent immunogenicity of the cells, it is possible to administer repeat doses without inducing a deleterious immune response.

For treating stroke, the neural stem cells can be administered intracerebrally. This can be achieved using stereotactic surgery.

In the Examples, patients receive CTX DP (20 million cells) by stereotaxic intra-striatal injection ipsilateral to the location of the MCA ischemic stroke.

Although the invention has been described in detail for purposes of clarity of understanding, certain modifications can be practiced within the scope of the appended claims. All publications, accession numbers, and patent documents cited in this application are hereby incorporated by reference in their entirety for all purposes to the same extent as if each were so individually denoted. To the extent more than one sequence is associated with an accession number at different times, the sequences associated with the accession number as of the effective filing date of this application are meant. The effective filing date is the date of the earliest priority application disclosing the accession number in question. Unless otherwise apparent from the context any element, embodiment, step, feature or aspect of the invention can be performed in combination with any other.

The invention is further described with reference to the following non-limiting examples.

### EXAMPLES

### Example 1: Trial Protocol—Pilot Investigation of Stem Cells in Stroke Phase II Efficacy (PISCES-II; NCT02117635)

Brief Summary:

The primary aim of this Phase II trial was to determine whether it is sufficiently likely that CTX Drug Product treatment at a dose level of 20 million cells improves the recovery in the use of the paretic arm in acute stroke patients to justify a subsequent larger prospectively controlled study.

This study evaluated the safety and efficacy of intracerebral CTX DP at a dose level of 20 million cells in patients with paresis of an arm following an ischemic middle cerebral artery (MCA) stroke. Eligible patients had no useful function of the paretic arm for a minimum of 28 days after the ischemic stroke (a modified NIH Stroke Scale (NIHSS) Motor Arm Score of 2, 3 or 4 for the affected arm).

| Condition or disease | Intervention/treatment | Phase |
|---|---|---|
| Ischemic Stroke | Biological: CTX DP | Phase 2 |
| Cerebral Infarction | | |
| Hemiparesis | | |
| Arm Paralysis | | |

Detailed Description:

Design: This Phase II efficacy trial was a multi-centre, open label, single arm, non-comparative design, administering a single dose of CTX cells 2 to 3 months post-ischemic stroke with follow-up over 12 months. The trial was overseen by an independent DSMB. The DSMB adjudicated at predetermined intervals whether a patient had satisfied the primary response criterion and whether the ongoing safety profile justified continuation or modification of the study.

At least 21 patients were enrolled to receive CTX DP (20 million cells) by stereotaxic intra-striatal injection ipsilateral to the location of the MCA ischemic stroke.

Pre-treatment selection of patients: Men and women, aged 40 or more, supratentorial ischemic stroke or a stroke with elements of both in an area perfused by the MCA (i.e., stroke due to ischaemia resulting in infarct located in the basal ganglia, internal capsule, or corona radiata or a stroke due to ischaemia resulting in infarction of part of the cerebral cortex).

Patients with a first stroke within the past 4 weeks (at time of consent) satisfying the following criteria: (a) modified NIHSS Motor Arm Score of 2 (some effort against gravity), 3 (no movement against gravity), or 4 (no movement) for the paretic arm post ischemic stroke; (b) clinical diagnosis of stroke confirmed by physician using neuro-imaging (computerised tomography or magnetic resonance imaging); and (c) a Score of 0 or 1 for test #2 of the ARAT at visit 1 and 2 using the affected arm.

Treatment: One patient was treated at one time. A single dose (20 million) of CTX DP cells was administered intracranially via stereotaxic neurosurgery.

Post-treatment follow-up: Patients were followed for 12 months post-implantation.

End-points: The primary endpoint of the trial was efficacy, using ARAT. Secondary endpoints were efficacy and safety. Outcome measures for efficacy included Fugl-Meyer, NIHSS, BI, and RFA. Safety was assessed by incidence of relevant adverse events and monitoring patient's general physical condition and clinical measures (temperature, pulse rate and rhythm, ECG, blood pressure, full blood count, liver function tests, serum urea, and electrolytes), immunological response, and concomitant medications at the 7 follow-up visits to the clinic in the first year after treatment.

Post-trial follow-up: Annual correspondence with family practitioners; Life-long follow-up for new diagnosis of cancer, site of primary tumour, and survival via National Cancer Registry.

Study Design:
Study Type: Interventional (Clinical Trial)
Actual Enrolment: 23 participants
Intervention Model: Single Group Assignment
Masking: None (Open Label)
Primary Purpose: Treatment
Official Title: A Phase II Efficacy Study of Intracerebral CTX0E03 DP in Patients With Stable Paresis of the Arm Following an Ischemic Stroke.
Study Start Date: June 2014
Actual Primary Completion Date: Aug. 16, 2017
Actual Study Completion Date: Aug. 16, 2017

Arms and Interventions:

| | |
|---|---|
| Experimental: allogeneic human | Biological: CTX DP |
| neural stem cell | 20 million cell dose administered by |
| CTX DP | surgery to the damaged area of the brain |
| human neural stem cell product, | Other Names: |
| single dose once only injection | CTX0E03 DP |
| | allogeneic human neural stem cell |
| | therapy product |

Outcome Measures:

Primary Outcome Measures
1. Action Research Arm Test (ARAT) [Time Frame: 3 months]

The primary outcome measure was a minimum 2 point improvement in the ARAT test number 2 (Yozbatiran et al., 2008).

Response was defined as a minimum improvement of 2 points in test number 2 of the ARAT (grasp a 2.5-cm3 block and move it from the starting position to the target end position) in the affected arm 6 months after injection of CTX DP. This represented an improvement from a pre-treatment state in which the patient was unable to grasp and reposition the block as required to a post-treatment state in which the patient could accomplish the task as specified within 60 seconds.

Secondary Outcome Measures
1. To assess the efficacy of intracerebral CTX DP in restoring upper limb function following an ischemic stroke using the ARAT [Time Frame: 12 months]
2. To assess the efficacy of intracerebral CTX DP in restoring function following an ischemic stroke using the Modified National Institutes of Health Stroke Scale (NIHSS) [Time Frame: 12 months]
3. To assess the efficacy of intracerebral CTX DP in restoring patient's functional independence following an ischemic stroke using the Rankin Focused Assessment (RFA) version of the modified Rankin Scale [Time Frame: 12 months]
4. To assess the efficacy of CTX DP in improving patient's ability to execute activities of daily living following an ischemic stroke using the Barthel Index (BI) [Time Frame: 12 months]
5. To assess the safety and tolerability of intracerebral CTX DP in patients following an ischemic stroke. Incidence of adverse events: monitoring of vital signs, temperature, pulse rate and rhythm, ECG, blood pressure, full blood count, liver function test, serum urea and electrolytes, CTX antibody screen. [Time Frame: 12 months]
6. To assess the efficacy of intracerebral CTX DP in restoring upper limb function following an ischemic stroke using the Fugl-Meyer Assessment [Time Frame: 12 months]

Eligibility Criteria

Ages Eligible for Study: 40 Years and older (Adult, Older Adult)

Sexes Eligible for Study: All

Accepts Healthy Volunteers: No

Inclusion Criteria:
Written informed consent or witnessed informed consent in the event that the patient is unable to sign informed consent due to paresis of the affected arm.
Supratenorial ischemic stroke
Male or female aged 40 years or more.
Stroke, at time of consent, satisfying the following criteria:
   Modified NIHSS Motor Arm Score of 2, 3 or 4 for affected arm visit 1 and visit 2.
   Clinical diagnosis of stroke confirmed by physician using neuro-imaging (CT or MRI).
   A Score of 0 or 1 for test 2 of the ARAT on day 28+7 and day 56+7 post-stroke using the affected arm.
Ability to comprehend verbal commands.
Eligible for neurosurgery including appropriate anatomical target for cell implantation.

Exclusion Criteria:
Prior history of stroke resulting in permanent moderate to severe disability (i.e., Rankin Scale greater than 2) (other than the presenting ischemic stroke).
Stroke due to hemorrhage.
History of neurological or other disease resulting in significant functional impairment of the paretic arm impairing potential ability to pick up, lift and place a 2.5 cm3 block (e.g., Parkinson's disease, motor neuron disease, arthritis, Dupuytren's contracture, or fixed anatomical abnormality).
Any contraindications to MRI including presence of a cardiac pacemaker (excluding MR-conditional cardiac pacemaker), metal fragments in eye, etc.
Uncontrolled blood pressure defined as systolic blood pressure ≥180 mm Hg or diastolic blood pressure ≥110 mm Hg (patients are only to be excluded if an initial value exceeding these limits is repeated on retesting over several days).
Patient with a severe comorbid disorder, not expected to survive more than 12 months.
Acute cardiovascular events other than the presenting ischemic stroke (e.g., myocardial infarction, recent coronary intervention for symptomatic cardiac disease) considered by the investigator or the anesthetist responsible for the patient to place the patient at increased anesthetic risk, 3 months prior to planned injection of CTX0E03 DP.
History of malignant disease (except for non-melanoma skin cancer) within the previous 5 years or any history of malignant brain tumours or brain metastasis.
Current treatment with tamoxifen.
Patients taking valproate drugs for any indication in whom it is not considered appropriate to discontinue the valproate for a period of one week prior and four weeks post neurosurgery. Patients in whom valproate is switched to an alternative agent during this period may be included.
Requirement for antiplatelets and/or anticoagulants including heparin, warfarin or other anticoagulants/medication that cannot be interrupted to allow surgery.
Requirement for intermittent (stop/start date from 1-month prior to and 3 month post CTX0E03 DP administration) use of oral antispasticity medications (oral antispasticity medications are acceptable if they have been taken regularly for at least one month prior to CTX0E03 DP administration).
A history of uncontrolled diabetes, e.g., history of hypoglycemic or hyperglycemic events requiring hospital admission over previous 6 months.
Females of childbearing potential (FOCBP) (or within 2 years of last menstrual cycle) must have a confirmed negative pregnancy test at the time of treatment and agree to use two reliable methods of contraception (e.g., oral contraceptive and condom, intra-uterine device (IUD) and condom, diaphragm with spermicide and condom) for the duration of this study.
Sexually active males with partners who are FOCBP must be willing to use a reliable method of contraception (e.g., barrier and spermicide or as described above) for the duration of this study.
Considered unlikely to be able to attend for all follow-up visits.
Organ transplant recipient.
In the opinion of the investigator, sustained consumption of alcohol or drugs at a level likely to be injurious to health.

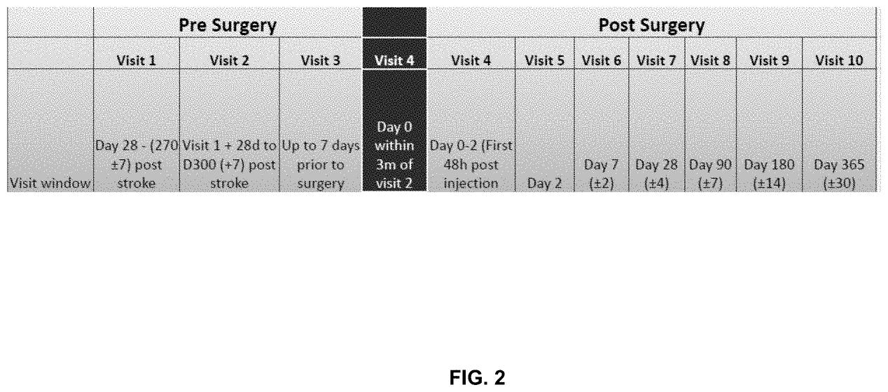

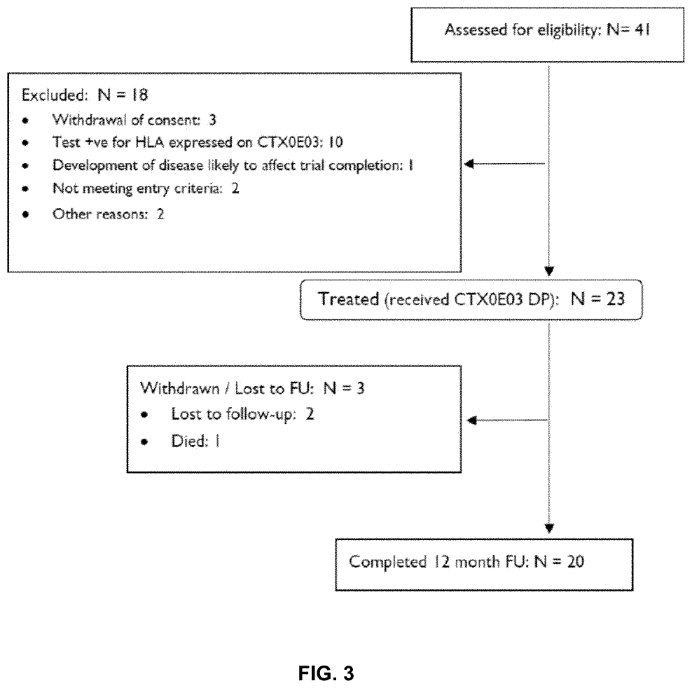

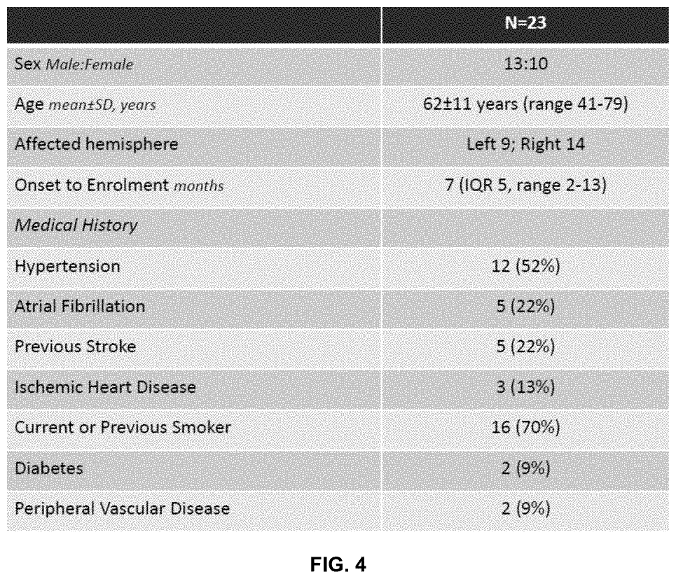

A number of the Figures assist with summarising the PISCES II Study. The Study Schedule (by visit) is shown in FIG. 2. The Recruitment and Patient Flow is shown in FIG. 3. The demographics of the participants are shown in FIG. 4.

Treatments

Treatments Administered

Patients meeting the eligibility criteria received an intracerebral implantation of 400 μL CTX0E03 DP at a nominal dose level of 20×10⁶ cells in sterile suspension on a single occasion. This represents an actual dose between 16 and 28×10⁶ cells in accordance with the product specification.

CTX0E03 DP was implanted under general anesthesia by a neurosurgeon experienced in stereotaxic intracerebral implantation. Stem cell delivery was performed using a technique used successfully in two previous clinical trials to implant stem cells intracerebrally by Kondziolka (Kondziolka et al., Cell Transplant 2004; 13(7-8):749-54.) and in ReNeuron's Phase I trial.

Identity of Investigational Product(s)

CTX0E03 DP is a formulation containing a human neural stem cell line developed by ReNeuron. CTX0E03 DP is an off-white, opaque, sterile suspension. It is composed of CTX0E03 cells at a passage of ≤37. The cells are formulated in HypoThermosol (HTS-FRS) at a concentration of 5×10⁴ viable cells/μL (range 4 to 7×10⁴ viable cells/μL). HTS-FRS is made up of ions, buffers, impermeants, a colloid, metabolites, and an antioxidant.

CTX0E03 DP was supplied, transported, and stored cryo-preserved at <−135° C. in a temperature controlled and monitored cryoshipper. Once the pharmacist was informed that CTX0E03 DP was released from quarantine and the patient was in the operating theatre ready for injection of CTX0E03 DP, the pharmacist thawed and dispensed the CTX0E03 DP for injection. This procedure ensured that the CTX0E03 DP was used within 3 hours from the time of thaw.

### Example 2: Trial Results—12 Month Functional Outcome After Intracerebral Implantation of Human Neural Stem Cells (PISCES II)

Baseline demographic and disease characteristics were generally representative of the population intended for the study. Overall, the majority of patients were white (95.65%), with similar numbers of males and females (13 M:10 F). The mean (SD) age was 62.39 (10.77) years. All patients were from the UK. The left arm was the most commonly affected (60.87%).

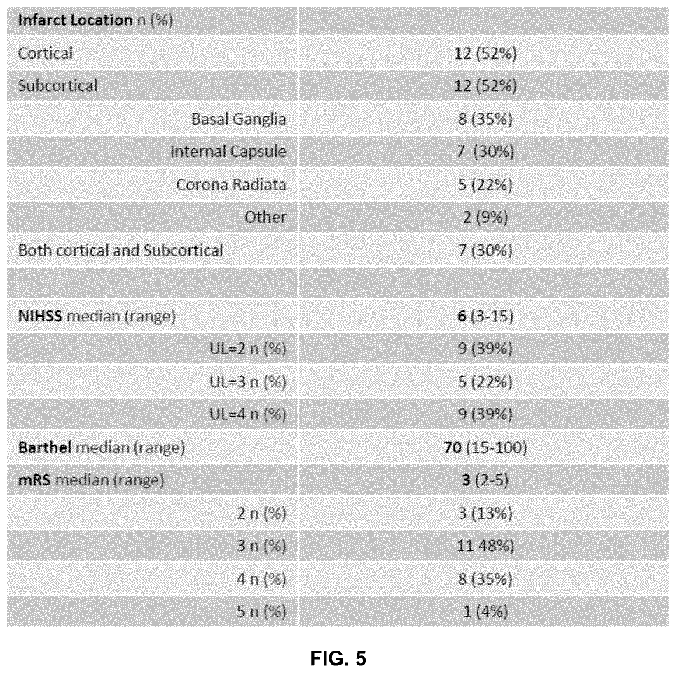

The results are summarised in FIGS. 5 to 11.

The Stroke Characteristics of the patients at Baseline is shown in FIG. 5.

FIG. 6 shows the PISCES II Responder Analysis: Primary and Secondary Measures. These data show that CTX0E031 treatment of the stroke patients led to at least a 2-point improvement in the ARAT Subtest #2 (grasp) in one patient after three months and in three patients after 6 months and 12 months. Furthermore, at least a six point improvement was observed in Total ARAT in two patients after just one month, 3 patients at 3 months, 4 at six months, and 5 at 12 months.

For the mRS, FIG. 6 shows that CTX0E03 treatment of the stroke patients led to at least one category of improvement in three patients after one month, and in seven patients after 3 months.

With respect to the Barthel Index, the results show that CTX0E03 treatment of the stroke patients led to an improvement of at least 9 points in the BI in six patients within one month, and in eight patients within three months.

The ARAT efficacy results are set out in FIG. 7. In the ARAT (Grasp) Test #2, ≥2 point improvement is seen in 3 responders, responding at 3, 6, and 12 months and with no relapse back to a lower score. For Total ARAT ≥6 point improvement is seen in 5 responders, responding at 1, 1, 3, 6, and 12 months.

The Median Total ARAT Response by Baseline NIHSS is depicted in FIG. 8. These Total ARAT Affected Limb results show improvements on ARAT observed in patients with residual movement of the affected arm (NIHSS 2 or 3 at baseline). These data reveal that the improvements in ARAT are observed in patients with residual movement of the affected arm (an NIHSS Motor Arm score of 2 or 3) at baseline. However, no improvement is seen in patients with no movement in the affected arm (NIHSS Motor Arm Score of 4).

FIG. 9 shows the modified Rankin Scale (mRS) Distribution: mRS improvement (12 m or final measured) compared to baseline in 7 (6 by 1 category, 1 by 2 categories); mRS unchanged in 14, worse (1 category) in 2. NB: 12 month mRS outcome determined 1 week prior to suicide in one subject therefore only one death at month 12 shown.

FIG. 10 shows the mRS by Baseline NIHSS: Improvement in mRS is greatest in patients with residual movement of the affected arm (NIHSS 2 or 3 at baseline).

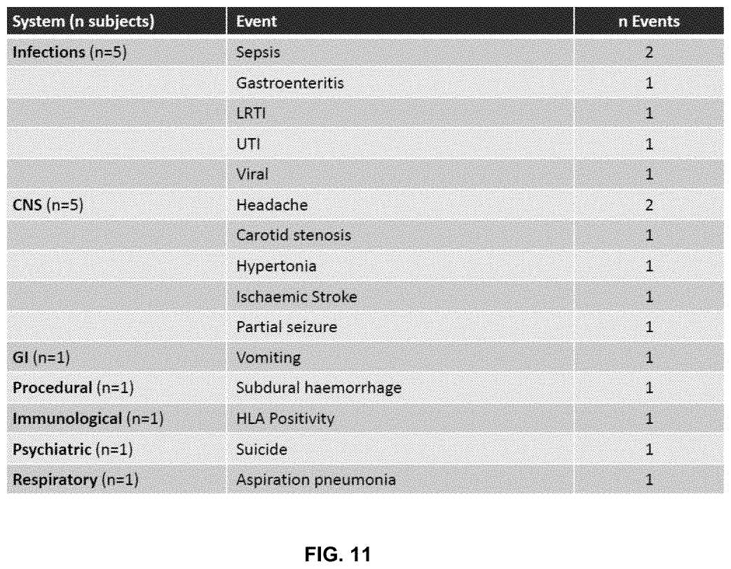

FIG. 11 shows Serious Adverse Events (SAEs) during follow-up.

### CONCLUSIONS

Overview

The results confirm the feasibility of subacute treatment with NSC implantation. The frozen cell product facilitated the multicentre trial. No cell-related safely issues were identified. Therapy three to six months after the stroke is acceptable to patients.

Functional gains were observed in sufficient numbers of patients to justify a further Phase 2b Trial.

Efficacy Conclusions

This study was designed to determine whether a sufficient proportion of patients experienced response of their paretic arm (improvement of ≥2 points in ARAT test 2) 3 months after implantation of CTX0E03 DP to justify a subsequent randomised study. The intent was to exclude response rates below 20% with 90% confidence at this 3-month timepoint. At the 3-month evaluation there was only 1 ARAT Test 2 responder and so the primary efficacy endpoint was not met. However, additional patients responded at 6 and 12 months suggesting that improvement continues beyond Day 90. Overall, 3/23 patients (13.64%) achieved an increase of at least 2 points at the last observation. One further patient responded at D180, could not grasp the 2.5-cm² block at Day 365, but had increased scores for the 5-cm² and 7.5-cm² blocks. This response rate is considered a demonstration of useful recovery of function in the paretic arm. Subgroup analysis showed that all 4 patients with a response were in the 14 patients with an NIHSS UL <4 at baseline, giving a response rate of 28.6% in the original planned population.

The Total ARAT score improved at the last observation in 7/23 patients with individual improvements ranging between 1 and 54 points. There were 5 (21.74%) responders (≥6 point improvement): 3 at Day 90, one more at Day 180, and another at Day 365.

Both the number of patients with an improvement, and the extent of improvement increased with time, and improvements were seen in all subscales of the ARAT Total Score compared to baseline. All patients with a response in the ARAT Total Score had NIHSS UL <4 at baseline. Therefore, the response rate within the original planned population of patients was 5/14 or 35.71%.

The mean NIHSS score improved by −1.43±1.40 in the planned patients at the Day 90 Analysis with further improvements at Day 180 and Day 365 giving −2.05±1.47 at Day 365. Improvements were seen in many different functional areas. In addition to recovery of arm function on NIHSS, the NIHSS leg function score improved to values better than any pre-treatment timepoint in 10/11 patients with sustained impairment pre-treatment.

The mRS score improved by −0.33±0.48 in the Day 90 Analysis with improvement maintained through Day 365. There were shifts to lower mRS compared to baseline in 9 patients overall, although 2 patients had worsened again by the last observation giving a response rate (improvement of at least one Grade) at any visit of up to 7/23 (35.00%) of patients. A response at Day 365 was more common in patients with a baseline NIHSS UL <4 (6/12, 50.00%) than those with baseline NIHSS=4 (1/8, 12.50%). Further exploratory inspection of subgroups, showed that this higher rate of response in patients with a baseline NIHSS UL Score <4 was broadly consistent in patients with and without mRS of 3-4 at baseline, and in patients treated less or more than 180 days after the presenting stroke.

The Barthel Index score improved by 7.38±11.79 in the Day 90 Analysis of the 21 planned patients with improvement maintained through Day 365. In the Day 365 analysis, 3/23 patients had the maximum score of 100 at baseline and so were not evaluable for improvement. In the remaining patients, improvements compared to baseline at post-treatment visits were seen in 17/20 patients, with 12 patients continuing to see improvement at Day 365, and improvements seen in most of the subscales. The response criteria (≥9 point improvement) was met in 12/17 patients (70.59%) at one or more visits: 8/17 patients at the Day 90 timepoint, with 7 responders at Day 180 and 8/17 responders (47.06%) at Day 365. 6 patients with the baseline BI >90 could not be assessed for response. Although subgroup analysis suggested a higher proportion of responders in patients with a baseline NIHSS UL=4, this was confounded by the ceiling effect which was mostly in the subgroup with NIHSS UL <4.

The FMA total motor and sensory score improved by 5.88±9.39 in the Day 90 analysis of the planned patients and continued to improve reaching a change of 8.00±13.89 at Day 365. There were 3/8 FMA responders in the Day 90 Analysis and 4/10 overall. This response was maintained or improved in 2 responders; one responder had worsened, dropping below the response criteria; and one Day 90 responder did not attend the Day 180 or Day 365 visit. Subgroup analysis did not demonstrate a difference in number of responders between the groups with baseline NIHSS UL=4, or <4.

### SELECTED REFERENCES

Banerjee, S., Williamson, D., Habib, N., Gordon, M., Chataway, J. (2011) Age and Ageing 40:7-13
Chung et al., Cell Stem Cell, 2, 113-117, 2008
Einstein, O., Ben-Hur, T. (2008) Arch Neurol 65:452-456
Gennaro (2000) Remington: The Science and Practice of Pharmacy. 20th edition, ISBN: 0683306472
Hassani Z, O'Reilly J, Pearse Y, Stroemer P, Tang E, Sinden J, Price J, Thuret S. “Human neural progenitor cell engraftment increases neurogenesis and microglial recruitment in the brain of rats with stroke.” PLoS One. 2012; 7(11):e50444. doi: 10.1371/journal.pone.0050444. Epub 2012 Nov. 21.
Hodges et al. Cell Transplant. 2007; 16(2):101-15
Horie, N., Pereira, N. P., Niizuma, K. Sun, G. et al. (2011) Stem Cells 29:274-285.
Kornblum, Stroke 2007, 38:810-816
Littlewood, T. D., Hancock, D. C., Danielian, P. S. et al. (1995) Nucleic Acid Research 23:1686-1690.
Miljan, E. A. & Sinden, J. D. (2009) Current Opinion in Molecular Therapeutics 4:394-403
Miljan E A, Hines S J, Pande P, Corteling R L, Hicks C, Zbarsky V, Umachandran, M, Sowinski P, Richardson S, Tang E, Wieruszew M, Patel S, Stroemer P, Sinden J D. Implantation of c-mycER TAM immortalized human mesencephalic-derived clonal cell lines ameliorates behavior dysfunction in a rat model of Parkinson's disease. Stem Cells Dev. 2009 March; 18(2):307-19
Pollock et al, Exp Neurol. 2006 May; 199(1):143-55.
Smith, E. J., Stroemer, R. P., Gorenkova, N., Nakajima, M. et al. (2012) Stem Cells http://www.ncbi.nlm.nih.gov/pubmed?term=smith%20stoemer%20gorenkova 30:785-796.
Stevenato, L., Corteling, R., Stroemer, P., Hope, A. et al. (2009) BMC Neuroscience 10:86
Stroemer, P., Patel, S., Hope, A., Oliveira, C., Pollock, K., Sinden, J. (2009) Neurorehabil Neural Repair 23: 895-909.
Their et al, “Direct Conversion of Fibroblasts into Stably Expandable Neural Stem Cells”. Cell Stem Cell. 2012 Mar. 20.

1. Neural stem cells for use in a method of treating ischemic stroke, wherein a single dose of the cells is administered as a treatment into the brain of a stroke patient having a modified NIHSS Motor Arm Score of 2 or 3, wherein the treatment improves motor function and alleviates disability within six months as determined by an increase of total ARAT score and/or a reduction in mRS of at least one category.

2. The neural stem cells of claim 1, wherein the treatment improves motor function within three months as determined by an increase of total ARAT score and/or a reduction in mRS of at least one category.

3. The neural stem cells of claim 1, wherein the treatment provides a reduction in mRS of at least one category within one month.

4. The neural stem cells of claim 1, wherein the patient has a modified NIHSS Motor Arm Score of 2 before treatment.

5. The neural stem cells of claim 1, wherein the patient has an ARAT #2 (grasp) test score of 0 or 1 before treatment.

6. The neural stem cells of claim 1, wherein the improved motor function remains after 12 months.

7. The neural stem cells of claim 1, wherein the increase of total ARAT score is at least six points.

8. The neural stem cells of claim 1, wherein a reduction in mRS of at least two categories is achieved within twelve months.

9. The neural stem cells for of claim 1, wherein the treatment improves function in the ARAT (Grasp) Test #2, by at least two points.

10. The neural stem cells of claim 8, wherein the improvement in ARAT (Grasp) Test #2 is provided 3, 6 or 12 months after treatment.

11. The neural stem cells of claim 1, wherein the cells are administered within four weeks from the stroke, within two months after the stroke, between two and twelve months after the stroke, between 3 and six months after the stroke, or between 6 and 12 months after the stoke.

12. The neural stem cells of claim 1, wherein the cells are administered between 3 and 6 months after the stroke.

13. The neural stem cells of claim 1, wherein the cells are administered into the brain by stereotaxic implantation.

14. The neural stem cells of claim 1, wherein between 16×10⁶ and 28×10⁶ cells are administered in the single dose.

15. The neural stem cells of claim 1, wherein 20 million cells are administered in the single dose.

16. The neural stem cells of claim 1, wherein the cells are from a neural stem cell line.

17. The neural stem cells of claim 1, wherein the cells are conditionally-immortalised.

18. The neural stem cells of claim 17, wherein the conditional-immortalisation is mediated by C-MycER.

19. The neural stem cells of claim 1, wherein the neural stem cells are CTX0E03 cells from the cell line having ECACC Accession No. 04091601.

20. A pharmaceutical composition comprising a single dose of neural stem cells and one or more pharmaceutically-acceptable excipients, formulated for implantation into the brain of a stroke patient having an NIHSS score of 2 or 3, for use in a method of treating ischemic stroke wherein the treatment improves motor function and alleviates disability within six months as determined by an increase of total ARAT score and/or an reduction of mRS of at least one category.

21. The pharmaceutical composition of claim 20, further comprising 6-hydroxy-2,5,7,8-tetramethylchroman-2-carboxylic acid (TROLOX®), Na⁺, K⁺, Ca²⁺, Mg²⁺, Cl⁻, H₂P0₄⁻, HEPES, lactobionate, sucrose, mannitol, glucose, dextron-40, adenosine and glutathione.

22. The pharmaceutical composition of claim 20, wherein the neural stem cells are CTX0E03 cells from the cell line having ECACC Accession No. 04091601.
